(19) United States
(12) Patent Application Publication
(10) Pub. No.: US 2008/0082117 A1
(43) Pub. Date: Apr. 3, 2008

(54) LANCING DEVICE

(75) Inventor: Christopher J. Ruf, Marietta, GA (US)

Correspondence Address: GARDNER GROFF GREENWALD & VILLANUEVA. PC, 2018 POWERS FERRY ROAD, SUITE 800, ATLANTA, GA, 30339, US

(73) Assignee: FACET TECHNOLOGIES, LLC, Kennesaw, GA (US)

(21) Appl. No.: 11/876,535

(22) Filed: Oct. 22, 2007

Related U.S. Application Data
(63) Continuation-in-part of application No. 10/987,591, filed on Nov. 12, 2004.
(60) Provisional application No. 60/519,232, filed on Nov. 12, 2003.

(30) Foreign Application Priority Data
Nov. 12, 2004 (US) PCT/US2004/037788

(51) Int. Cl. A61B 17/32

(52) U.S. Cl. 606/182

(57) ABSTRACT

A lancing device for blood sampling, having a depth-adjustment mechanism including a chassis or throttle plate movable within a housing, and a lancet carrier mounted to the chassis or throttle plate. Repositioning the chassis or throttle plate within the housing varies the penetration depth of the lancet.

### CROSS REFERENCE TO RELATED APPLICATIONS

This application is a continuation-in-part of U.S. patent application Ser. No. 10/987,591, filed Nov. 12, 2004, which claims the benefit of U.S. Provisional Patent Application Ser. No. 60/519,232, filed Nov. 12, 2003, which applications are hereby incorporated herein by reference in their entireties.

### TECHNICAL FIELD

The present invention relates generally to medical devices and procedures, and more particularly to lancing devices for the collection and/or analysis of samples of blood or other bodily fluids, and to related methods of manufacture and use of lancing devices.

### BACKGROUND OF THE INVENTION

Lancing the skin of a human or animal subject is commonly carried out to obtain a sample of blood or other body fluid, as for example in blood glucose monitoring by diabetics and in blood typing and screening applications. A disposable lancet having a sharp tip may be loaded into a reusable lancing device having a spring-biased or otherwise energized drive mechanism for moving the lancet through a lancing sequence, for convenience and safety.

In many instances, it is desirable to allow the user to vary the depth of penetration of the lancet tip into the skin during a lancing procedure. To minimize pain and healing time, the shallowest penetration depth necessary to generate adequate sample size is generally sought. Various depth control mechanisms and methods are known, including the provision of lancets of differing sizes, adjustment of lancet openings on the lancing device, and varying the stroke of travel of the lancet.

It is to the provision of an improved lancing device and depth control mechanism that the present invention is primarily directed.

### SUMMARY OF THE INVENTION

Briefly described, example embodiments of the present invention include an improved lancing device that is convenient, compact, and permits a user to accurately adjust the lancing depth of a lancet. The improvements of the present invention preferably increase convenience and minimize pain for the user, thereby encouraging more frequent testing and ensuring compliance with the subject's prescribed testing regimen.

In the various example embodiments shown and described herein, the present invention provides a lancing device having an internal throttle plate or chassis comprising the drive mechanism and lancet carrier, which is controllably repositionable relative to the outer housing of the device to vary the depth of penetration by the lancet. In this manner, the lancet traverses a constant stroke length relative to the chassis, and depth control is accomplished by adjustment of the position of the chassis within the housing.

In one aspect, an example lancing device according to the present invention includes an external housing having a first end and a second end, the first end defining an opening. The lancing device further includes a chassis movable within the housing between a forward position toward the first end of the housing and an aft position away from the first end of the housing. The lancing device further includes a lancet carrier mounted to the chassis for engaging a lancet. The lancing device further includes a drive mechanism for propelling the lancet carrier along a lancing stroke. The lancing device further includes an adjustment member operable to move the chassis between the forward position and the aft position, and thereby provide depth adjustment, the forward position corresponding to a deeper penetration depth, and the aft position corresponding to a shallower penetration depth.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWING FIGURES

FIG. 1 is a top view of a lancing device according to an example embodiment of the present invention.

FIG. 2 is a side view of the lancing device of FIG. 1 in partial cross-sectional view, taken at section line A-A.

FIG. 3 is a plan view of a lancing device according to another example embodiment of the present invention.

FIG. 4 is a top perspective view of a lancing device according to still another example embodiment of the present invention.

FIG. 5 is an exploded perspective view of the lancing device of FIG. 4.

FIG. 6 is a bottom perspective view of the lancing device of FIG. 4.

FIG. 7 is a plan view of a lancing device according to another example embodiment of the present invention.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

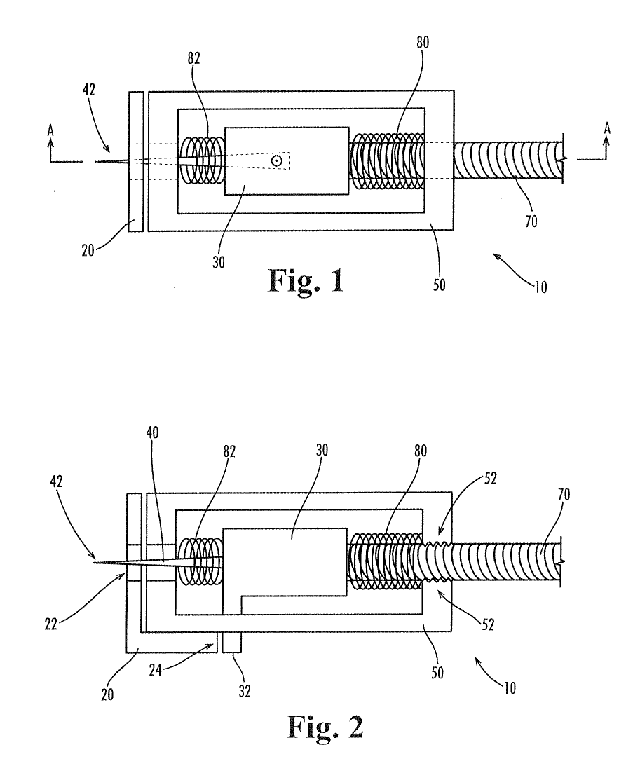

With reference to the example embodiment of the present invention shown in FIGS. 1 and 2, a lancing device 10 includes an outer housing 20 (partially shown) having an opening for permitting the tip of the lancet to pass therethrough in its extended or lancing position, to penetrate the subject's skin at a lancing site pressed against the housing outside the opening. A throttle plate or chassis 50 is mounted within the housing 20. A lancet carrier 30 is coupled to the chassis 50, and receives a replaceable lancet 40, or alternatively comprises an integral lancet portion.

At least one drive spring 80 engages the lancet carrier 30 to propel the carrier and a lancet engaged therein along a lancing stroke, from a retracted or energized position within the housing 20 to its extended position. Optionally, at least one return spring 82 is provided for returning the lancet carrier from the extended position to a neutral position wherein the lancet tip is shielded from contact within the housing to prevent inadvertent needle-sticks. In the depicted embodiment, the drive and return springs 80, 82 are coil springs arranged in an in-line configuration, coaxial with the lancet's stroke of travel, and engaged between the chassis 50 and opposed ends of the lancet carrier 30. In alternate embodiments, the springs can be different types of springs, and/or can be laterally offset or otherwise configured, or another form of drive mechanism can be provided such as a solenoid, pneumatic drive, or other mechanical, electromechanical or electromagnetic drive means.

The lancing depth (i.e., the distance the tip of the lancet 40 extends outwardly of the housing 20 in the lancet's extended position) is adjusted by linearly moving the chassis 50 within the external housing 20. In the depicted embodiment of the invention, the throttle plate or chassis 50 moves forward and backward relative to the housing 20 by movement along a positioning screw comprising a threaded rod 70, or other positioning implement. The chassis 50 includes a threaded hole 52 for receiving corresponding threads of the rod 70. A knob or other actuator connected to the threaded rod 70 extends externally of the housing 20, such that rotation of the knob turns the positioning screw to advance or retract the chassis 50 within the housing. As the chassis 50 is moved towards the opening 22 in the housing 20, more of the lancet 40 is exposed when the lancet is in its advanced position, such that the lancet penetrates deeper into the skin. The chassis 50 can be moved away from the opening 22 to provide for a shallower lancing depth.

The lancing stroke of the lancet 40 and carrier 30 is preferably constant relative to the chassis 50, such that the length of the stroke remains unchanged regardless of the position of the chassis as it is moved relative to the housing to provide lancing depth adjustment. In example embodiments, a positional adjustment mechanism such as a screw-driven rack is provided for varying the position of the lancet and/or the drive mechanism within the housing of the lancing device. In further example embodiments, the position or spring constant (stiffness) of the return spring and/or the drive spring of the lancet drive mechanism can be varied to provide depth control.

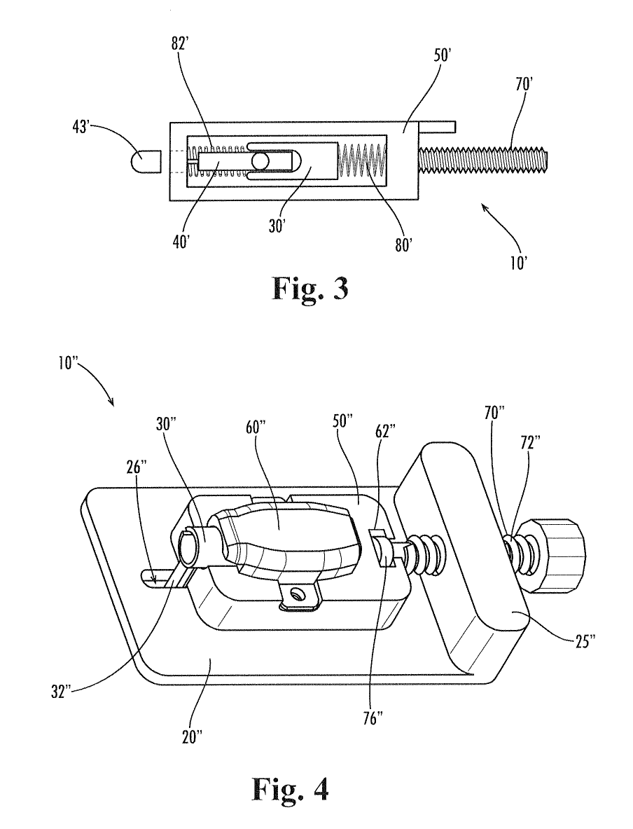

FIG. 3 shows another example embodiment of a lancing device having a depth control mechanism incorporating a movable throttle plate or chassis 50′ that is positionally adjustable within an unshown housing via actuation of a positioning screw 70′ to provide lancing depth adjustment. A lancet carrier 30′ releasably engages a lancet 40′ having a removable protective endcap 43′, and is driven along a lancing stroke by a drive spring 80′ and a return spring 82′. The throttle plate 50′ optionally defines an internal chamber for receiving and guiding the lancet carrier 30′ along a linear path, and defining front and/or back stops that move in tandem with the throttle plate as its position is adjusted, to limit the forward and backward travel of the lancet carrier and define a constant stroke of traverse. Optionally, the housing can also include front and/or back stop surfaces for engaging a corresponding protrusion of the lancet carrier, to limit the linear travel of the chassis within a predetermined range. Alternatively, no stop surfaces are provided, wherein a user can position the chassis as desired at any point within the housing.

Referring now to FIGS. 4-6, another example embodiment of an improved lancing device 10″ according to the present invention is shown. Generally, the lancing device 10″ comprises a housing 20″ (shown in part), a lancet carrier 30″ for receiving a lancet (not shown) therein, a movable lancet chassis 50″, and a positioning screw 70″. The housing preferably includes a one slot 26″ for receiving a complementary alignment protrusion 32″ of the lancet carrier 30″. Alternatively or additionally, the housing includes two additional slots 27″, 28″ for receiving complementary alignment protrusions or feet 54″, 55″ of the chassis 50″. The housing 20″ also includes a rear wall 25″ having a threaded hole 29″ for receiving the positioning screw 70″ therethrough.

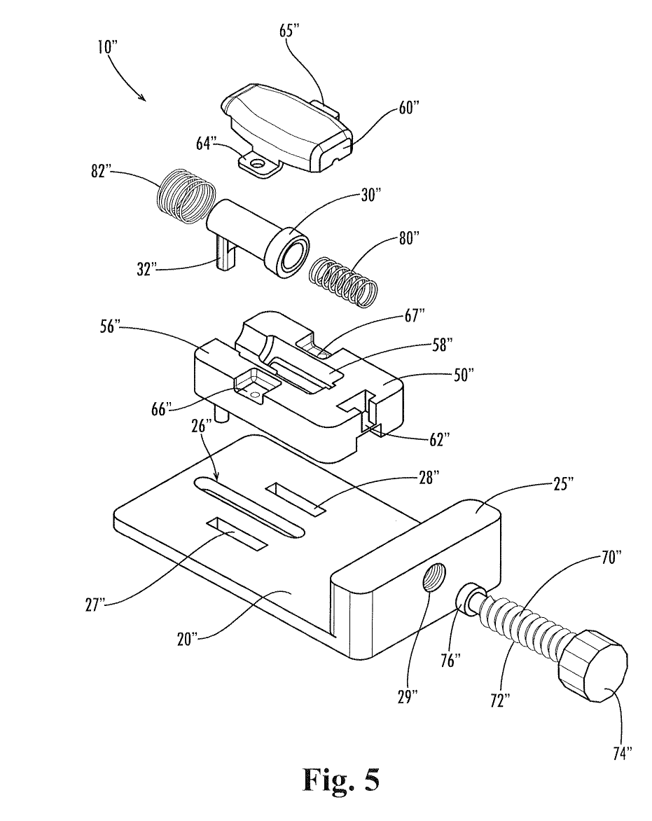

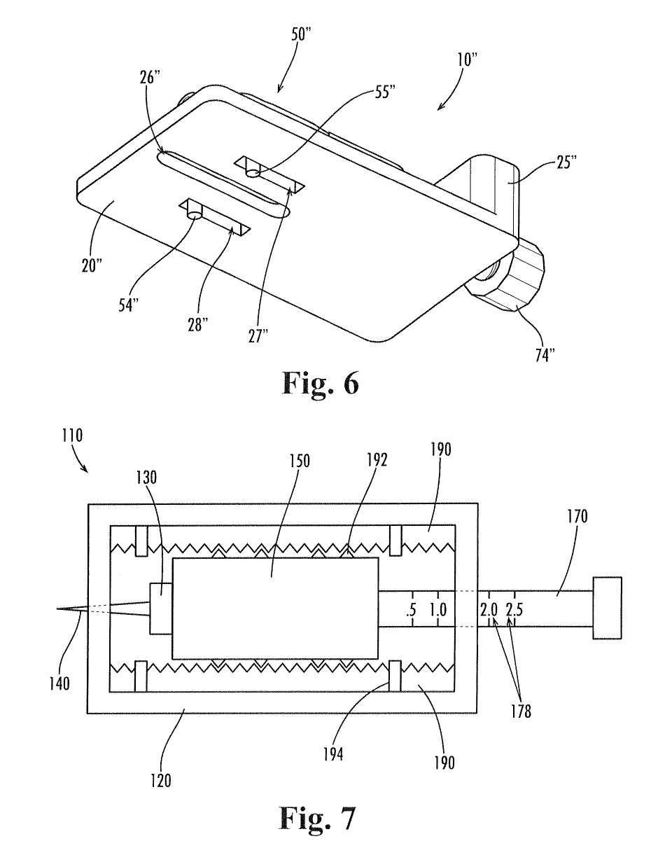

The chassis 50″ generally comprises a chassis body 56″, a lancet carrier cavity 58″ for receiving the lancet carrier 30″ therein, a lancet carrier cover 60″, and a coupling 62″ for engaging the positioning screw 70″, as seen in FIG. 5. The chassis 50″ is slidably and repositionably mounted to the housing 20″ and is aligned thereon by extending the feet 54″, 55″ into corresponding slots 27″, 28″ (FIG. 6). The lancet carrier 30″ is seated within the lancet carrier cavity 58″ and is secured therein by the lancet carrier cover 60″. The lancet carrier cover 60″ can include one or more fastening prongs 64″, 65″ for engagement with corresponding recesses 66″, 67″ of the chassis 50″ to secure the cover to the chassis. The lancet cover 60 also constrains the drive spring 80″ and return spring 82″ within the lancet cavity 58, such that the springs remain captively engaged with the lancet carrier 30″. The lancet carrier 30″ is permitted to slide linearly within the lancet cavity 58, wherein the lancet carrier is capable of completing a lancing stroke (i.e. moving the lancet from a retracted/charged position to an extended/lancing position). The positioning protrusion 32″ of the lancet carrier 30″ extends through the slot 26″ in the housing 20″ for aligning and guiding the lancet carrier along a linear path as the carrier completes a lancing stroke, for energizing or cocking the drive mechanism, and/or for engaging a release or triggering mechanism to initiate the lancing sequence.

The positioning screw 70″ comprises a threaded body portion 72″, an optional handle or knob 74″, and a coupling knob 76″, as best seen in FIG. 5. The positioning screw 70″ is inserted through the threaded hole 29″ of the housing 20″, wherein the threaded body portion 72″ engages the complementary threads of the housing. The coupling knob 76″ is inserted into engagement with the chassis coupling 62″, such that the knob 76″ is permitted to freely turn therein, while prohibiting lateral or linear movement between the same. As a user advances or retracts the positioning screw 70″ through the threaded hole 29″ in the housing 20″, the chassis 50″ is linearly shifted away from or towards the rear wall 25″ of the housing, respectively, thereby controlling the lancing depth of a lancet (not shown) positioned within the lancet carrier 30″. As seen in FIG. 6, the linear travel of the chassis 50″, and ultimately the range of depth control for the lancet, can be limited by the length of the slots 26″, 27″, 28″ in the housing 20″. As the chassis 50″ is linearly shifted along the housing 20″, the alignment feet 54″, 55″ of the chassis and the carrier protrusion 32″ of the lancet carrier 30″ prevent linear travel beyond the length of the corresponding slots 26″, 27″, 28″. The length of the slots can vary as desired to increase or decrease the range of depth control of the improved lancing device 10″.

Typically, a user desires for a particular lancet to penetrate their skin or other tissue only as far as needed for the collection of the blood or other bodily fluid. However, the deeper a particular lancet penetrates a user's tissue, the more painful the lancing becomes. Therefore, a user utilizing the improved lancing device of the present invention to collect blood or other bodily fluids is able to control the piercing depth of a lancet as desired. In operation of the embodiment of FIGS. 4-6, a user can advance the positioning screw 70″ into the housing 20″ to shift the chassis 50″ away from the rear wall 25″ and towards the lancing end of the housing for deeper lancing. The user can continue to shift the chassis 50″ in this direction as desired, until the alignment feet 54″, 55″ contact the distal end of the slots 27″, 28″. As the chassis 50″ reaches the distal end of the slots, the chassis and lancet carrier 30″ are fully advanced. Conversely, the user may withdraw the positioning screw 70″ from the housing to shift the chassis 50″ towards the rear wall 25″ for shallower lancing. The user may continue to move the chassis 50″ in this direction as desired until the alignment feet 54″, 55″ contact the proximal end of the slots 27″, 28″, wherein the chassis and carrier 30″ are fully retracted. In example embodiments, the lancing stroke of the lancing device 10″ remains constant regardless of the positioning of the chassis 50″ within the housing 20″, and the penetrating depth of a lancet is controlled in the manner described above. Alternatively, the lancing stroke can also be modified to work in conjunction with the depth control mechanism of the improved lancing device 10″.

In another example embodiment, as seen in FIG. 7, a lancing device 110 comprises a housing 120, a lancet carrier 130, a lancet 140, a movable chassis 150, a chassis plunger 170, and a toothed rack or rail 190. In this embodiment, the chassis 150 can be moved in relation to the housing 120 by advancing or retracting the chassis plunger 170 into/out of the housing, to provide lancing depth adjustment. The plunger 170 can be a substantially rigid rod that is coupled to the chassis 150. In preferred embodiments, the plunger 170 includes indicia 178 for visually or otherwise indicating a particular penetration depth of the lancet 140. The chassis 150 includes one or more positioning teeth 192 for engaging and interacting with the rack 190. The teeth 192 can be formed of a resilient material or biased by a spring or otherwise, to permit the chassis 150 to linearly slide along the rack 190, while substantially securing the chassis in place once a user has identified a particular chassis position. Additionally, one or more stops 194 can be added to the rack 190 to limit the linear travel of the chassis 150 as desired.

While the invention has been described with reference to preferred and example embodiments, it will be understood by those skilled in the art that a variety of modifications, additions and deletions are within the scope of the invention, as defined by the following claims.

What is claimed is:

1. A lancing device comprising:
an external housing having a first end and a second end, the first end defining an opening;
a chassis movable within the housing between a forward position toward the first end of the housing and an aft position away from the first end of the housing;
a lancet carrier mounted to the chassis for engaging a lancet;
a drive mechanism for moving the lancet carrier relative to the chassis, along a lancing stroke; and
an adjustment member operable to selectively reposition the chassis between the forward position and the aft position, and thereby provide lancing depth adjustment, the forward position corresponding to a deeper lancing depth, and the aft position corresponding to a shallower lancing depth.

2. The lancing device of claim 1, wherein the adjustment member is rotationally actuated to move the chassis between the forward position and the aft position.

3. The lancing device of claim 1, wherein the drive mechanism comprises a drive spring and a return spring.

4. The lancing device of claim 1, wherein the lancing stroke has a constant stroke distance regardless of the positional adjustment of the chassis relative to the housing.
